(19) United States
(12) Patent Application Publication
(10) Pub. No.: US 2022/0362570 A1
(43) Pub. Date: Nov. 17, 2022

(54) NERVE STIMULATION

(71) Applicant: Philip Pemberton, Bromsgrove (GB)

(72) Inventor: Philip Pemberton, Bromsgrove (GB)

(21) Appl. No.: 17/764,443

(22) PCT Filed: Sep. 28, 2020
(86) PCT No.: PCT/IB2020/059040

§ 371 Date: Mar. 28, 2022

(30) Foreign Application Priority Data
Sep. 26, 2019 (GB) 1913902.1

(51) Int. Cl. A61N 2/00; A61N 2/02; A61G 13/12; A61M 16/00

(52) U.S. Cl. CPC A61N 2/006; A61N 2/02; A61G 13/1215; A61M 16/0003; A61M 2205/057

(57) ABSTRACT

A device and method for providing mechanical ventilation of a user is described. In an embodiment the device comprises at least two metallic coils, each coil configured to be placed adjacent to a phrenic nerve of the user; and a stimulation unit for providing an electric current to the metallic coils, and wherein the current stimulates the phrenic nerve to induce tetanic contractions of a diaphragm muscle of the user to regulate the user's breathing. This provides a ventilation whilst reducing the rehabilitation time post ventilation for a user due to lower muscle wasting of the diaphragm.

### FIELD OF INVENTION

The current invention relates to nerve stimulation and in particular to a nerve stimulating device to provide mechanical ventilation of a user.

### BACKGROUND OF THE INVENTION

Mechanical ventilation has been the cornerstone of respiratory support in intensive care for the past half century. Most intensive care units ventilate patients using intermittent positive pressure ventilation (IPPV). This involves the use of positive pressure across a patient's airway to cause insufflation of the lungs with oxygen enriched gas. Although ventilator settings may target volumes, pressures or time as the main trigger for cycling on and off, the main mechanism of ventilation is generally the same.

This mode of ventilation by positive pressure across an airway is in contrast to that used by a healthy person spontaneously breathing. In healthy individuals, normal breathing involves active contraction of the diaphragm muscle, which creates a negative intrathoracic pressure within the pleural cavity. This negative pressure draws air into the lungs as transpulmonary pressure causes the lungs to expand, and air to flow from atmosphere into the alveoli. Whilst negative pressure ventilation exists that mimics this process, it is performed without diaphragm stimulation, and so is subject to the issues described below.

Although mechanical ventilation is clearly a lifesaving intervention, how this therapy is applied to patients can affect mortality and morbidity outcomes. Tidal volumes, airway pressures, duration of ventilation and even the act of mechanical ventilation itself have all been shown to influence patient prognosis. The latter is thought to be the main cause of ventilator induced diaphragmatic dysfunction (VIDD)—a period of diaphragmatic mechanical silence triggering a rapid decrease in diaphragm size and strength.

The occurrence of VIDD is associated with increased morbidity and mortality in ventilated patients. A somewhat underappreciated phenomenon is that when placed on a mechanical ventilator, the diaphragm muscle stops actively contracting. In health, this unique circumstance is the only time in a person's life that this will happen (in a healthy person the diaphragm continuously contracts subconsciously whether awake or asleep to maintain adequate ventilation). Just as the quadriceps or biceps muscles are seen to waste during prolonged periods of inactivity this diaphragmatic disuse atrophy follows a similar pathophysiology to peripheral skeletal muscle disuse atrophy, although it may occur at an accelerated rate. Whilst exercise continues to be the main intervention proposed as a countermeasure, it has been suggested that it is the combination of neural activation as well as muscle contraction that are required to protect against disuse atrophy. It would therefore follow that VIDD could be attenuated by exercising the diaphragm during this period of disuse.

Current methods to exercise the diaphragm in intensive care patients rely on a degree of patient participation which is not feasible in the majority of patients undergoing mandatory ventilation due to delirium or levels of sedation employed to facilitate tolerance of the endotracheal breathing tube. Paradoxically, it is during this period that the greatest proportion of diaphragm strength is lost.

In addition to being non-physiological, current methods of mechanical ventilation can directly damage the lungs. In patients with ‘stiff’ lungs high airway pressures, large tidal volumes and high respiratory rates are often employed when trying to achieve adequate ventilation. Partial or full ventilation by means of active contraction of the diaphragm in these patients would help to mitigate the amount of positive pressure required and potentially the amount of damage incurred.

Direct electrical phrenic nerve pacing is a recognised technique for diaphragm stimulation, using a pacemaker to provide electrical stimulation. However pacemaker insertion requires surgical implantation and is generally reserved for patients with high spinal cord lesions and central hypoventilation syndrome. Transcutaneous electrodes pose a risk of tissue injury, activation of cutaneous pain receptors, skin irritation and failure through insecure fixation. Percutaneous electrodes inserted directly into the diaphragm reduce the risk of thermal tissue injury but alternatively risk injury to abdominal organs, bleeding and infection. Transvenous phrenic nerve pacing has been demonstrated in animals but requires the insertion of a central venous catheter and is subject to varying efficacy with time.

Magnetic stimulation of the phrenic nerves has been used for measurement of diaphragm strength for more than two decades. In this instance, stimulating coils are manually held in place anterolaterally at each side of the neck over the left and right phrenic nerves in a co-operative, immobile subject. A single, supramaximal magnetic pulse is produced that depolarises the phrenic nerves and causes a single maximal contraction of the diaphragm. The pressure across the diaphragm during this contraction (transdiaphragmatic pressure (TwPdi)) is used as a measure of the strength of the diaphragm. The devices used for this measurement are able to produce adequate single stimuli but are currently not able to produce the frequency of stimulation required to produce tetanic, sustained contractions of the diaphragm, which are necessary to induce negative pressure ventilation.

Transcranial magnetic stimulation is a treatment modality licensed for the treatment of depression in addition to other disorders. The frequencies used in this type of stimulation could theoretically produce tetanic contractions of the diaphragm but the stimulator units are unable to power the two coils simultaneously required for paired left and right phrenic nerve stimulation. In addition to the frequency required, the stimulation length, limited rest period and train duration needed to stimulate diaphragm contractions would cause current devices to overheat within a short time period.

The present invention aims to at least ameliorate the aforementioned disadvantages by providing device that is able to produce tetanic, sustained contractions of the diaphragm through magnetically stimulated, bilateral, phrenic nerve stimulation.

### SUMMARY

According to a first aspect of the present invention, there is provided a device for providing mechanical ventilation of a user, said device comprising: at least two metallic coils, each coil configured to be placed adjacent to a phrenic nerve of the user; and a stimulation unit for providing an electric current to the metallic coils and wherein the current stimulates the phrenic nerve to induce tetanic contractions of a diaphragm muscle of the user to regulate the user's breathing.

The present invention provides a stimulation device that can ensure that wasting of the diaphragm muscle is minimised during periods where it cannot function as normal, such as during mechanical ventilation. Through active contractions of the diaphragm the present invention can both regulate a user's breathing and reduce muscle wasting of the diaphragm. By providing external electromagnetic stimulation the present device can be easily applied quickly and without the use of surgery to implant a pacemaker or the like. This allows the device to be applied to patients as needed, and can be adjusted as required.

In a preferred embodiment, the stimulation unit may further comprise a current pulse generator for supplying current pulses to each coil simultaneously, such that a time-varying magnetic field is induced on each coil to stimulate the phrenic nerve; and a control unit for controlling one or more of the amplitude, pulse width, frequency and/or train duration of the current supplied to each coil.

Optionally or preferably the current pulse generator may be configured to supply current pulses of up to 30 Hz frequency.

The stimulation unit further may further comprise a trigger unit capable of receiving a signal from an external ventilator unit, said signal providing data of a ventilation state of the ventilator unit, and wherein the stimulation unit synchronises supply of the current pulses with the signal. The signal may supply the data at a frequency of at least 100 Hz. Synchronising the supply of electrical pulses with the action of the mechanical ventilator allows the tetanic contractions to be synced with the pressure changes occurring within the thoracic cavity, allowing prescription of concentric or eccentric contractions of the diaphragm as desired, and potentially limiting ventilator induced damage to internal organs, in particular the airways and lungs. In this way the combination of the present invention and the ventilator is replicating the natural breathing process more closely.

In a further example, the current pulse generator may further comprise one or more capacitors to store electrical energy for supply to the coils, said one or more capacitors connected to a transformer; and a thyristor, wherein the current from the one or more capacitors is discharged via the thyristor. This provides a safety back-off in the event of overheating or overstimulation being detected.

A collar may be provided, said collar being configured to house the metallic coils and to position said coils adjacent to the phrenic nerve of a user when worn over a neck of the user. Ideally there should be some degree of flexibility in the exact position of the coil to allow ‘fine tuning’ as necessary although this is likely to be in the order of millimetres.

The collar may comprise a semi-fixed device including a pillow for supporting a user and one or more moveable arms for positioning the metallic coils into position on the user. This can allow the device to be heavier, with the weight of the coils and any cooling system typically necessitating a device weight of 10 kg+, such as 12 kg. By utilising a pillow, this can support the weight of the user when prone and support the user. Arms either side of the pillow can then position the coils in the desired location.

Accordingly, in embodiments, the coils may be housed within a one or more heads of collar, each head located at the end of one of the moveable arms. The moveable arms comprise an upper arm coupled to the head and a lower arm coupled between the upper arm and the pillow, and wherein the upper arm, lower arm and head are independently moveable to position the head against the phrenic nerve of a user, when in use.

A mattress support may be coupled to the collar for supporting the collar in place relative to a bed to reduce movement of the collar when the bed is non-horizontal. This allows the device to be used when the user is lying in bed in a non-horizontal position, without gravity pulling the device down the bed.

Typically the pillow may mouldable to a user using either a vacuum or a pneumatic inflation device. Accordingly, the pillow may substantially surrounds a neck and head of a user either by design or by inflating the pillow to surround and support the user as needed.

Each coil may be fixed to the collar via a pocket located in a body of the collar, such that each coil can be attached to, or detached from, the pocket. Accordingly, the coils may be fixed within the collar. It can also be appreciated that the size of the collar may be adjustable. This can ensure that the coils are placed correctly for patients of differing neck size and physiology. The collar may incorporate a magnetic shield to partially shield the magnetic field produced by each coil. This can ensure that the stimulation is correctly directed to minimise unintended effects to surrounding tissues and nerves. A cover may be used for the collar, said cover being disposable after use allowing the collar to be reused for different users.

In an embodiment, each coil may be made from a plurality of windings stacked in a plurality of layers, and wherein the layers are aligned. In one example, the shape of each layer may form a figure of eight pattern.

Additionally or alternatively, each coil may have a concave contour.

In one example, cooling fluid may circulates through or around the coils to transfer heat from the coils to the fluid. The fluid may be electrically insulated from electrical components of the device by at least two layers of containment. An alarm may be triggered in an event of leakage of the fluid through a primary containment layer. This can alert physicians or nurses to a failure in the unit and the risk of overheating. It can be appreciated that the alarm may act to automatically cut-off supply of electrical signals to the coils. In order to measure temperature, the coils may incorporate a thermistor or thermocouple for continuous monitoring of coil temperature.

Typically the stimulation unit can be electrically connected to each coil via an electrically insulated cable.

According to a second aspect of the present invention, there is provided a method of stimulating a phrenic nerve of a user to induce tetanic contractions of a diaphragm muscle of the user, said method comprising the steps of: aligning the coils of the device according to any embodiment of the first aspect, such that each coil is placed adjacent to the phrenic nerve of the user; and supply electrical pulses to said coils to induce tetanic contractions of the diaphragm muscle.

There may be provided a computer program, which when run on a computer, causes the computer to configure any apparatus, including a circuit, controller, sensor, filter, or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software implementation may be an assembly program.

The computer program may be provided on a computer readable medium, which may be a physical computer readable medium, such as a disc or a memory device, or may be embodied as a transient signal. Such a transient signal may be a network download, including an internet download.

These and other aspects of the invention will be apparent from, and elucidated with reference to, the embodiments described hereinafter.

### BRIEF DESCRIPTION OF DRAWINGS

Embodiments will be described, by way of example only, with reference to the drawing, in which

FIG. 1 illustrates a diaphragm stimulation device according to an embodiment of the present invention;

FIG. 2a illustrates a diaphragm stimulation device according to an alternative embodiment of the present invention;

FIG. 2b shows an alternative view of the device of FIG. 2a;

FIG. 3a shows a front view of an alternative embodiment of the device of FIG. 2 when in use; and

FIG. 3b shows a rear view of FIG. 3a.

It should be noted that the Figures are diagrammatic and not drawn to scale. Relative dimensions and proportions of parts of the Figures have been shown exaggerated or reduced in size, for the sake of clarity and convenience in the drawings. The same reference signs are generally used to refer to corresponding or similar feature in modified and different embodiments.

### DETAILED DESCRIPTION OF THE DRAWINGS

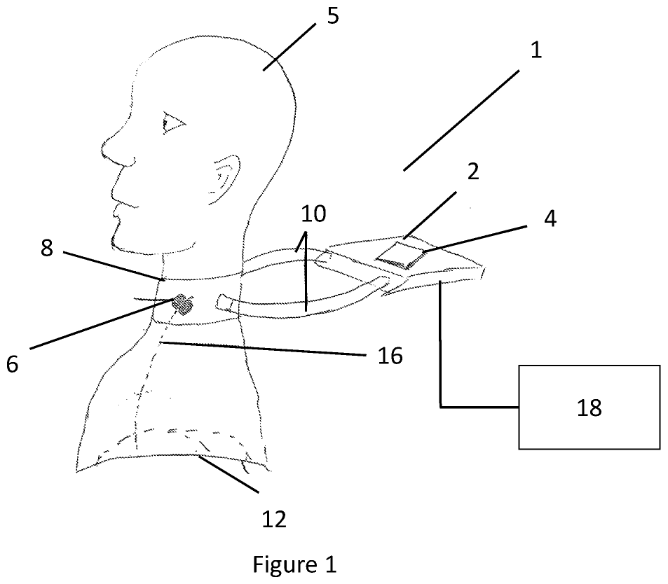

FIG. 1 shows a schematic of a device 1 according to one embodiment, comprising a simulation unit 2, control unit 4, and a collar 8. The collar 8 houses a plurality of coils 6, as shown in FIG. 1. The collar 8 and coils 6 may be connected to the stimulation unit 2 by means of single or paired flexible cables 10. Cables 10 may be electrically insulated and may be detachable from the collar 8 or fixed, or detachable only from the stimulation unit 2.

The stimulation unit 2 is configured to deliver high current electrical pulses to the coils 6 such that a time-varying magnetic field is induced in the coils 6. The purpose of providing such a magnetic field is to induce Eddy currents (by virtue of Faraday's Law) that penetrate the skin, soft tissue and bone and in particular act to stimulate a patient's phrenic nerves 16 to produce tetanic, sustained contractions of the patient's 5 diaphragm muscle 12. In the shown embodiment, at least two coils 6, placed either side of the patient's 5 neck are used, to cause paired depolarisation of the left and right phrenic nerves 16 respectively.

The left and right phrenic nerves 16, arising largely from the anterior primary ramus of C4 with contributions from C3 and C5 are branches of the cervical plexus that provide the motor innervation to the diaphragm 12. After becoming superficial at the posterior border of the sternomastoid muscle the nerves run on the anterior surface of the scalenus anterior muscle, deep to sternomastoid, caudally and medially between the subclavian vessels and under the clavicle to enter the thoracic cavity.

In one embodiment, the collar size is adjustable and may have varying angles of contour to accommodate for different shape and sizes of patient 5 necks. This allows the collar 8 to fit securely around the patient's 5 neck, as well as enabling fine tuning of the final position of the coils over the phrenic nerves 16. The latter facilitates accurate delivery of phrenic nerve specific stimulations at the lowest power output possible whilst also being removable to allow access neck access, turning, washing or any other procedure required.

In some arrangements, the collar 8 may incorporate magnetic shielding to allow a more focused magnetic field and to minimize the co-stimulation of unwanted structures (muscle tissue and other nerves). The design of the collar 8 and magnetic field shielding may be such that the induced current targets nerves 16 as they travel between the sternomastoid muscle and the thoracic cavity. According to another embodiment, the coils 6 may be fixed using pockets for the coils 6 in the body of the collar 8, such that they can be attached and detached as needed, or they may be inbuilt and non-removable. In embodiments, the collar 8 sits inside of, or is attached to, a disposable element that will facilitate single use for each patient.

In order to produce sustained contractions of the diaphragm 12, the current pulse delivered to the coils must be of sufficient intensity, frequency, pulse width and train duration. The control unit 4 within the stimulating unit 4 allows an operator to manipulate the power setting for each stimulation, the pulse width, frequency and train duration as required.

To ensure that a high current pulse of sufficient intensity is supplied, the stimulation unit 4 may store high intensity electrical energy in one or more capacitors that are connected to a transformer, which is then rapidly discharged (over ˜100 μs) through the stimulating coil 6 via a thyristor. The one or more capacitors may store voltages of up to 6 kV. The total discharge time may be up to 1 ms with a rise time of <150 μs. The mode of current discharge is such that a rapidly alternating magnetic field is produced that is able to induce an electric field in excitable tissue in its path. In other embodiments, the stimulating unit 2 can produce stimulations at a range of frequencies up to approximately 30 Hz. For example, for ventilation, the stimulation may be 63 Joules, supplied at 30 Hz for 1000 ms with a 2 second rest. This is a rate of 20 pulses supplied per minute. This gives approximately a 10 A current per 1 second stimulation. Typical train durations may be 1 minute or longer, and continuous application can also be used.

In some embodiments, the coils 6 may be made from a plurality of windings overlaid on top of each other to form layers. The wires of the coil 6 may be made from copper or any other material of low resistance, and may be solid or hollow. The shape of each winding may be a figure of eight, oval, clover leaf shape or other suitable geometries. Such features of the coil 6 can act to increase the magnetic field strength further, since the size of this magnetic field strength is a function of the number of windings in the coil 6, the type of coil 6 (single circular or figure of eight) and the current passing through it. For example, a figure of eight pattern allows for increased magnetic field strength by combining the magnetic fields where the coil wires cross, with a peak intensity ˜40% of the coil radius from the surface plane.

These properties of electromagnetic stimulation can obviate the need for high surface currents that sensitize nociceptors that plague electrical stimulation. In addition, the magnetic field produced this way is such that the stimulating coil 6 need not even touch the skin surface, although the shorter the distance between the coil 6 and the conductor to be stimulated (i.e. the patient) the greater will be the strength of the magnetic field that reaches it. Electromagnetic stimulation of the phrenic nerves therefore represents a completely non-invasive technique for diaphragm stimulation.

In other embodiments, the coil shape has a concave contour that allows it to be positioned against the patient's 5 neck in the optimal position to facilitate induction of current in the phrenic nerves 16. In other arrangements, the coils 6 can be linked by cables 10 such that one trigger is able to activate all coils 6 simultaneously and cause paired depolarisation of the left and right phrenic nerves 16.

In one embodiment, the coils 6 are cooled using a fluid compound that circulates through or around the coils transferring heat from the coils 6. This compound may undergo changes in physical state (i.e. may be solid until sufficient energy has been absorbed from the coils to change it into the liquid state) or remain in the liquid state. The circulating coolant may be insulated from the electrical components of the device 1 with two levels of redundancy so in the event of failure of the primary containment an alarm can be triggered and the device 1 will be shut off before further damage occurs. In some embodiments, the coils 6 may incorporate a thermistor or thermocouple for continuous temperature monitoring. This will allow manual or automatic adjustment of the cooling liquid temperature or flow speed as required to maintain the surface temperature of the coils 6 at the desired temperature.

In some embodiments, the stimulating unit 2 may incorporate a trigger that is able to receive a signal from a ventilator 18 in order to trigger the stimulation. This allows continuous synchronisation of the stimulations with the ventilator cycles of the ventilator 18. This signal transmission may be via RS232, HDMI, BNC, modem, fibre-optic, RF or other interface as required to communicate with a variety of manufacturers hardware.

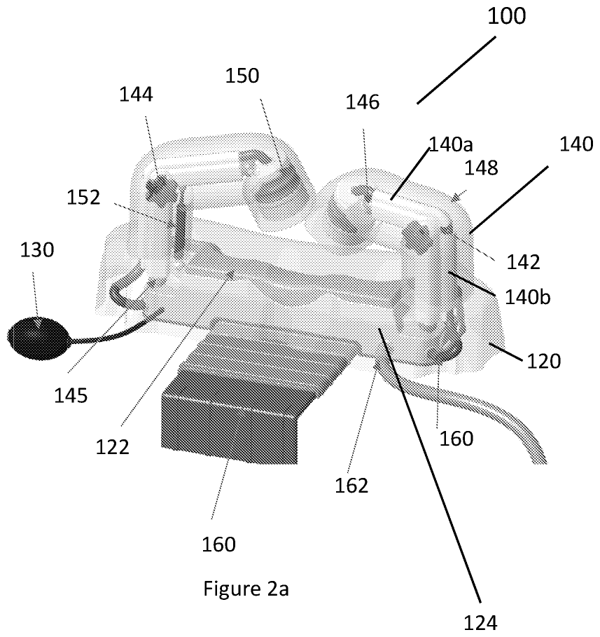

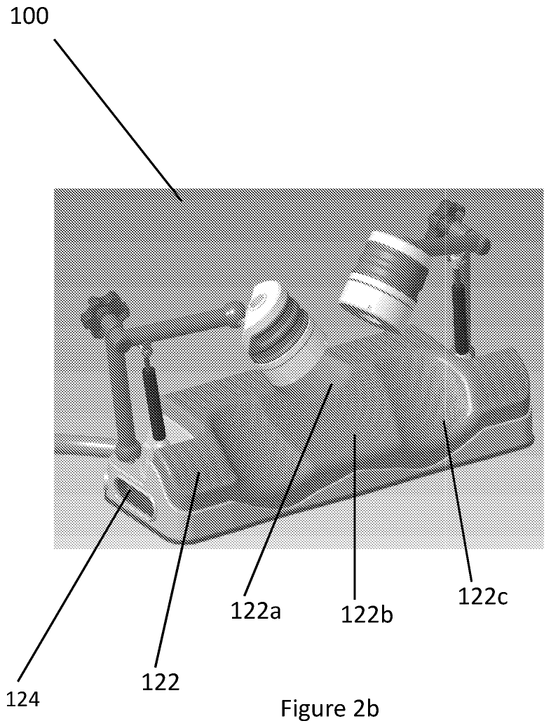

FIG. 2a shows another exemplary embodiment 100 of the present invention. In this embodiment, there is provided a separate unit 120, instead of a collar 8 in FIG. 1. The unit 120 comprises a neck pillow 122 supported on a base 124 to allow the user to rest the head, instead of the user wearing the collar 8. FIG. 2b shows the front view of the unit comprising the neck pillow 122. The neck pillow 122 comprises a neck contour 122a to receive a neck of a user, a back contour 122b for supporting the user's back and shoulder supports 122c.

The neck pillow 122 may be inflatable using a hand pump 130 to inflate the pillow 122 to a desired stiffness according to the weight and support level required by the user. As can also be seen from FIGS. 2a and 2b, two arms 140 extend from the opposite ends of a base of the unit. The arms comprise two sections, an upper arm 140a, and a lower arm 140b. The upper and lower arms 140a, 140b are connected by an elbow pivot 142 for adjusting the position of each arm so that a head 150 at the end of each arm adjacent to a user's phrenic nerve. The head 150 may also be attached to a ball joint 146 to allow the head to be rotated into position. A gas strut 152 (or a pneumatic strut or similar) can be used to maintain the height of the upper arm 140a. The strut enables the weight of the coils to be supported by the arm.

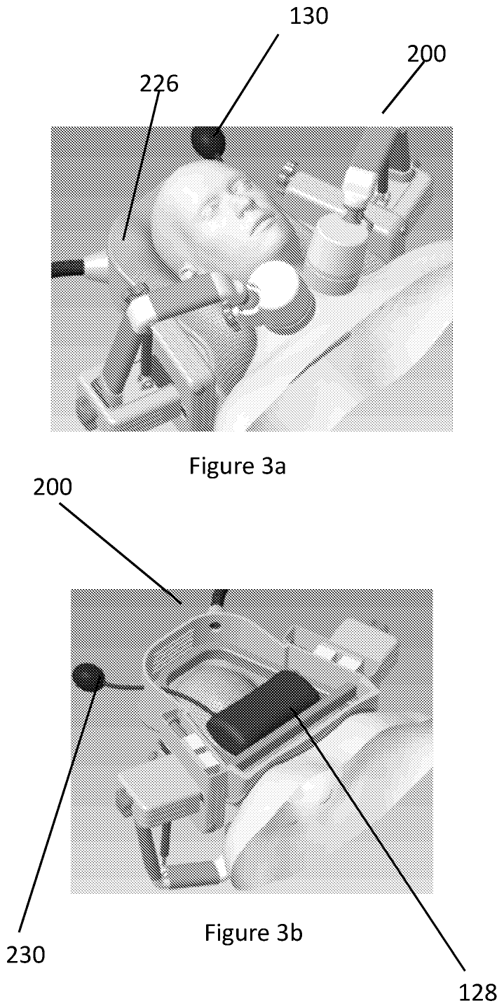

Accordingly, each arm has three individual joints: one at the base 145, one in the middle 144, and one at the top 146. The base 145 and the top 146 joints are enabled via the use of lower and upper ball joints, respectively, as shown in FIG. 2a. The middle joint 142 is locked with a locking screw during use. The joints allow manual positioning of the coils around the patient's neck when the user's head is rested onto the neck pillow, as shown in FIG. 3a.

The arms 140 and head 150 are covered in a polythene cover that aids cleaning. The cover may be disposed after every use. The coil heads 150 each house a magnetic coil that is coupled via a cable 160, 162 to a stimulation unit of a ventilator 18 that supplies electric current to the coil such that the coil stimulates a phrenic nerve of a user when positioned adjacent to said nerve on a user.

Each head and arm comprises a cooling system that can utilise either solid or liquid cooling systems. In the solid system the head 150 incorporates a heatsink that buffers any temperature rise on the surface of the coil.

In embodiments using a liquid system, this can be active or passive. A passive system incorporates a static liquid (or compound that can hold a solid or liquid form dependant on its temperature) that sits in the coil head and absorbs the heat produced by the coil. An active system incorporates a liquid that is circulated through the coil head and back down the linking cable to a coolant unit that continually cools the fluid.

In embodiments, the pillow itself may also be used as a store or reservoir for the coolant.

In a further embodiment, the system may also include an adjustable mattress support 160 that hooks over the top of the bed to resist any tendency for gravity dependant movement of the apparatus 100 when used on patients in a ‘head up’ position.

In yet another embodiment as shown in FIG. 3, the unit 200 comprises a full head pillow 226 instead for the neck pillow 120. FIG. 3b shows the back view of the full head pillow 226. As previously, the neck pillow 120 and/or the full head pillow 226 can be moulded to the user using a vacuum to maintain its shape. This will necessitate a fairly rigid pillow. Alternatively, the pillow itself can be soft and incorporate a pneumatic device that when inflated can add further support to the patients neck and/or head, as shown in FIG. 3b. The latter can be achieved by means of an inflatable neck lift 228 such as the one shown in FIG. 3b. The neck lift 228 can be inflated manually using a balloon pump 230.

The software written for the device may be such that it allows interrogation of the ventilator 18 for the required trigger for stimulation. This trigger cycles on and off of the mechanical ventilator cycle. As this setting may be changed numerous times per day the software assesses this information for each stimulation and using it to automatically cycle on and off the stimulation trains. All such settings may be manually adjustable by the user.

From reading the present disclosure, other variations and modifications will be apparent to the skilled person. Such variations and modifications may involve equivalent and other features which are already known in the art of nerve stimulation, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination. The applicant hereby gives notice that new claims may be formulated to such features and/or combinations of such features during the prosecution of the present application or of any further application derived therefrom.

For the sake of completeness it is also stated that the term “comprising” does not exclude other elements or steps, the term “a” or “an” does not exclude a plurality, a single processor or other unit may fulfill the functions of several means recited in the claims and reference signs in the claims shall not be construed as limiting the scope of the claims.

1. A device for providing mechanical ventilation of a user, said device comprising:
at least two metallic coils, each coil configured to be placed adjacent to a phrenic nerve of the user; and
a stimulation unit for providing an electric current to the metallic coils, and wherein the current stimulates the phrenic nerve to induce tetanic contractions of a diaphragm muscle of the user to regulate the user's breathing.

2. The device of claim 1, wherein the stimulation unit further comprises:
a current pulse generator for supplying current pulses to each coil simultaneously, such that a time-varying magnetic field is induced on each coil to stimulate the phrenic nerve; and
a control unit for controlling one or more of the amplitude, pulse width, frequency and/or train duration of the current supplied to each coil.

3. The device of claim 2, wherein the current pulse generator is configured to supply current pulses of up to 30 Hz frequency.

4. The device of claim 2, wherein the stimulation unit further comprises a trigger unit capable of receiving a signal from an external ventilator unit, said signal providing data of a ventilation state of the ventilator unit, and wherein the stimulation unit synchronises supply of the current pulses with the signal.

5. The device of any claim 4, wherein the signal supplies the data at a frequency of at least 100 Hz.

6. The device of claim 2, wherein the current pulse generator further comprises:
one or more capacitors to store electrical energy for supply to the coils, said one or more capacitors connected to a transformer; and
a thyristor, wherein the current from the one or more capacitors is discharged via the thyristor.

7. The device of claim 1, further comprising a collar, said collar configured to house the metallic coils and to position said coils adjacent to the phrenic nerve of a user when worn over a neck of the user.

8. The device of claim 7, wherein the collar comprises a pillow for supporting a user and one or more moveable arms for positioning the metallic coils into position on the user.

9. The device of claim 8, wherein the coils are housed within a one or more heads of collar, each head located at the end of one of the moveable arms.

10. The device of claim 9, wherein the moveable arms comprise an upper arm coupled to the head and a lower arm coupled between the upper arm and the pillow, and wherein the upper arm, lower arm and head are independently moveable to position the head against the phrenic nerve of a user, when in use.

11. The device of claim 8, further comprising a mattress support coupled to the collar for supporting the collar in place relative to a bed to reduce movement of the collar when the bed is non-horizontal.

12. The device of claim 8, wherein the pillow is mouldable to a user using either a vacuum or a pneumatic inflation device.

13. (canceled)

14. The device of claim 7, wherein each coil is fixed to the collar via a pocket located in a body of the collar, such that each coil can be attached to, or detached from, the pocket.

15. The device of claim 7, wherein the collar incorporates a magnetic shield to partially shield the magnetic field produced by each coil.

16. (canceled)

17. The device of claim 7, further comprising cooling fluid that circulates through or around the coils to transfer heat from the coils to the cooling fluid.

18. The device of claim 17, wherein the cooling fluid is electrically insulated from electrical components of the device by least two layers of containment,
and wherein an alarm is triggered in an event of leakage of the fluid through a primary containment layer.

19. (canceled)

20. The device of claim 17, wherein the cooling fluid is stored within a reservoir, said reservoir located within the collar.

21. The device of claim 1, wherein each coil is made from a plurality of windings stacked in a plurality of layers, and wherein the layers are aligned and a shape of each layer forms a figure of eight pattern.

22. (canceled)

23. The device of claim 1, wherein each coil has a concave contour.

24. (canceled)

25. (canceled)

26. A method of stimulating a phrenic nerve of a user to induce tetanic contractions of a diaphragm muscle of the user, said method comprising the steps of:
aligning the coils of the device according to claim 1 such that each coil is placed adjacent to the phrenic nerve of the user; and
supply electrical pulses to said coils to induce tetanic contractions of the diaphragm muscle.
